(19) United States
(12) Patent Application Publication
(10) Pub. No.: US 2020/0269071 A1
(43) Pub. Date: Aug. 27, 2020

(54) POSITIONING HEAD FRAME FOR GAMMA KNIFE

(71) Applicant: SHENZHEN OUR NEW MEDICAL TECHNOLOGIES DEVELOPMENT CO., LTD., Shenzhen City (CN)

(72) Inventors: Wenhao SUN, Shenzhen City (CN); Liang WANG, Shenzhen City (CN); Ziheng NIE, Shenzhen City (CN)

(21) Appl. No.: 16/756,830

(22) PCT Filed: Oct. 16, 2017
(86) PCT No.: PCT/CN2017/106340

§ 371 Date: Apr. 16, 2020

(51) Int. Cl. A61N 5/10

(52) U.S. Cl. CPC A61N 5/1084; A61N 2005/1097; A61N 2005/1055

(57) ABSTRACT

A positioning head frame for gamma knife is provided. The positioning head frame includes a base ring, a plurality of vertical rods circumferentially arranged on the base ring and a plurality of head nails in threaded connection with the plurality of vertical rods. At least one of the base ring and the vertical rod is insulated.

### TECHNICAL FIELD

The present disclosure relates to the field of medical equipment, and more particularly to a gamma knife positioning head frame.

### BACKGROUND

In the treatment of head gamma knife, it is necessary to use a gamma knife positioning head frame to locate the lesion on a patient's head and then tomography is performed to the lesion by CT (Computed Tomography) or MRI (Magnetic Resonance Imaging) to reconstruct a three-dimensional morphology of the skull, lesion and surrounding tissue. The gamma knife positioning head frame includes a metal base ring, a plurality of metal vertical rods arranged on the metal base ring in the circumferential direction, and a head nail in a threaded connection with the top of the metal vertical rod. During application, the metal base ring encircles the head, the head nail is adjusted to abut against the target position of the cranium of the patient, and lesion positioning can be realized.

Currently, the components of the gamma knife positioning head frame are all made of a conductive metallic material. During an MRI scan, the positioning head frame forms a closed loop and generates a large induced electromotive force which is eventually dissipated in the form of thermal energy, thereby causing the head nail to heat up and causing a burn of the head of the patient when it is serious. In the prior art, this technical problem is solved by improving the nail structure. Specifically, the head nail is divided into two parts of a detachable connection and an insulating layer is provided at the joint of the two parts to disable the closed loop formed by the positioning head frame.

The inventors have discovered that there are at least the following problems in the prior art.

The insulation layer is easily worn under frequent use which causes the failure of disabling the closed circuit, and that head tack is arranged in segments increases the probability of damage.

### SUMMARY

The embodiments of the present disclosure provide a gamma knife positioning head frame, and the above technical problem can be solved. The detailed technical solutions are as follows:

A gamma knife positioning head frame including a base ring;

a plurality of vertical rods disposed circumferentially on the base ring; and

a plurality of head nails in threaded connection with a plurality of said uprights;

the base ring and/or the vertical rod are insulated.

In one possible design, the positioning head frame further includes an insulation sleeve;

the insulating sleeve is detachably connected with the top of the vertical rod; and

an inner wall of the insulating sleeve is provided with internal threads for being in threaded connection with the head nail.

In one possible design, the insulating sleeve is clamped to the top of the vertical rod.

In one possible design, at least one elastic clip is arranged on a side wall of the insulating sleeve;

a clamping hole is formed in the top of the vertical rod, and a clamping groove matching with the at least one elastic clip is arranged on an inner wall of the clamping hole; and

the insulating sleeve is detachably clamped in the clamping hole by the at least one elastic clip.

In one possible design, a fixing hole is arranged in the top of the vertical rod and

the insulating sleeve is in interference fit with the fixing hole.

In one possible design, the top of the vertical rod is provided with a mounting hole and a limiting hole, axes of the mounting hole and a limiting hole are perpendicular to each other and the insulating sleeve is in clearance fit with the mounting hole; and

the positioning head frame further includes a plurality of fastening screws in threaded connection with the limiting hole and configured for abutting against the insulating sleeve after passing through the limiting hole.

In one possible design, the top of the vertical rod is provided with a large diameter hole and a small diameter hole which are in coaxial communication;

an inner wall of the large diameter hole is provided with inner threads rotating in the same direction as inner threads of the insulating sleeve, and the insulating sleeve is in threaded connection with the large diameter hole; and

a diameter of the small diameter hole is smaller than an outer diameter of the insulating sleeve and is larger than an outer diameter of the head nail.

In one possible design, the insulating sleeve includes a cylindrical section and a force application section connected to the cylindrical section.

In one possible design, the force application segment is prepared by forming two opposed planes on a cylinder and

a diameter of the cylinder is larger than a diameter of the cylindrical section.

In one possible design, the force application section is provided with a fool-proof structure.

In one possible design, the base ring is wholly or partially insulated and/or the vertical rod is wholly insulated or partially insulated.

In one possible design, the vertical rod and/or the base ring are wholly or partially insulated by using engineering plastic.

The technical solutions provided by the embodiments of the present disclosure may include the following benefits.

According to the gamma knife positioning head frame provided by the embodiment of the present disclosure, the conductive loop of the positioning head frame can be disabled by insulating the base ring and/or the vertical rod and it is ensured that the induced current generated by the induced electromotive force is not conducted to the head nail, so that the head nail is prevented from heating up, and the head of the patient is prevented from being burned. According to the embodiment of the present disclosure, the conductive loop is disabled by insulating the base ring and/or the vertical rod, it can not only effectively prevent the head nail from heating up but can also avoid the head nail structure from being changed and thus the durability of the head nail is ensured.

### BRIEF DESCRIPTION OF THE DRAWINGS

In order to describe the technical solutions in the embodiments of the present disclosure more clearly, the following briefly introduces the accompanying drawings required for describing the embodiments. Apparently, the accompanying drawings in the following description show merely some embodiments of the present disclosure, and a person of ordinary skill in the art may also derive other drawings from these accompanying drawings without creative efforts.

FIG. 1 is a structural schematic view of a gamma knife positioning head frame in accordance with embodiments of the present disclosure;

FIG. 2-1 is an exploded view of a vertical rod, a head nail and an insulating sleeve in accordance with embodiments of the present disclosure;

FIG. 2-2 is an assembled view of the vertical rod, the head nail and the insulating sleeve in accordance with embodiments of the present disclosure;

FIG. 3-1 is a structural schematic view of a first type of insulating sleeve in accordance with embodiments of the present disclosure;

FIG. 3-2 is a cross-sectional view of a vertical rod matching with the first type of insulating sleeve structure in accordance with embodiments of the present disclosure;

FIG. 4 is a cross-sectional view of a vertical rod matching with a second type of insulating sleeve structure in accordance with embodiments of the present disclosure;

FIG. 5 is a cross-sectional view of a vertical rod matching with a third type of insulating sleeve structure in accordance with embodiments of the present disclosure;

FIG. 6 is a cross-sectional view of a vertical rod matching with a fourth type of insulating sleeve structure in accordance with embodiments of the present disclosure;

FIG. 7-1 is an isometric view of the second type of insulating sleeve in accordance with embodiments of the present disclosure; and

FIG. 7-2 is a top view of the second type of insulation sleeve in accordance with embodiments of the present disclosure.

### DETAILED DESCRIPTION ABOUT THE REFERENCE NUMBERS

1 base ring,
2 vertical rod,
2a1 clamping hole, 2a2 clamping groove,
2b fixing hole,
2c1 mounting hole, 2c22c2 limiting hole,
2d1 large diameter hole, 2d2 small diameter hole,
3 head nail,
4 insulating sleeve,
4a elastic clip,
401 cylindrical section, 402 force application section, and 4021 fool-proof structure

### DETAILED DESCRIPTION

Unless defined otherwise, all technical terms used in the disclosed embodiments have the same meaning as commonly understood by one of ordinary skill in the art. In order to make the objects, technical solutions and advantages of the present disclosure more comprehensible, embodiments of the disclosure will be described in further detail below in conjunction with the accompanying drawings.

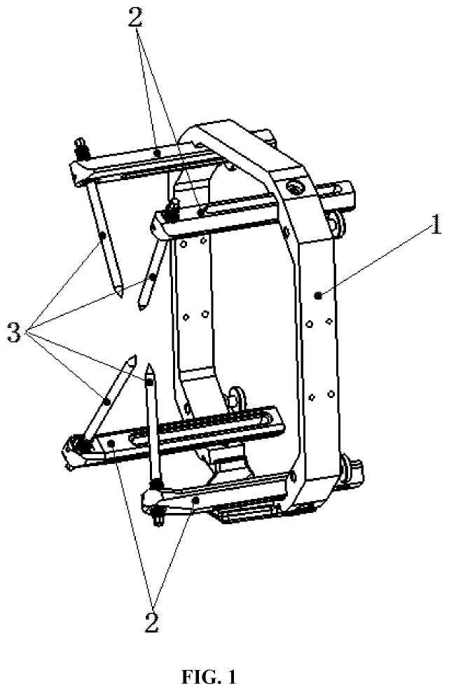

As shown in FIG. 1, the embodiment of the present disclosure provides a gamma knife positioning head frame including a base ring 1, a plurality of vertical rods 2 circumferentially disposed on the base ring 1 and a head nail 3 in a threaded connection with the plurality of vertical rods 2, and wherein the base ring 1 and/or the vertical rod 2 are insulated.

According to the gamma knife positioning head frame provided by the embodiment of the present disclosure, the conductive closed loop of the positioning head frame can be disabled by insulating the base ring 1 and/or the vertical rod 2 and it is ensured that the induced current generated by the induced electromotive force is not conducted to the head nail 3, so that the head nail 3 is prevented from heating up and the head of the patient is prevented from being burned. As can be seen, the conductive loop is disabled by insulating the base ring 1 and/or the vertical rod 2 according to the embodiments of the present disclosure, and it can not only effectively prevent the head nail from heating up but can also avoid the head nail structure from being changed and thus ensures the durability of the head nail.

Furthermore, it should be noted that when the gamma knife positioning head frame provided by the embodiment of the present disclosure is applied, the base ring 1 encircles the patient's head and its distance toward the patient's head is adjusted by screwing the head nail 3 so that the head nail 3 abuts against patient's head at the cranium target position and the positioning of the lesion on patient's head is realized. It can be understood that the base ring 1 takes the patient's head as a center point and is disposed around it, the vertical rod 2 is disposed on the base ring 1 in the axial direction of the base ring 1 and the head nail 3 is disposed along the radial direction of the base ring 1 to ensure that the head nail 3 can abut against the patient's cranium.

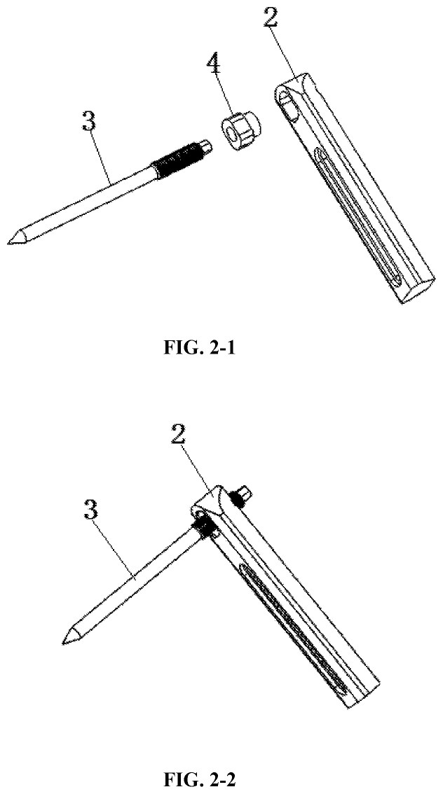

As shown in FIGS. 2-1 and 2-2, the positioning head frame also includes an insulating sleeve 4. The insulating sleeve 4 is detachably connected with the top of the vertical rod 2; and the inner wall of the insulating sleeve 4 is provided with internal threads to be in threaded connection with the head nail 3.

In the gamma knife positioning head frame provided by the embodiment of the present disclosure, 4a the conductive loop of the positioning head frame can also be disabled by arranging the insulating sleeve 4 as a connecting part between the vertical rod 2 and the head nail 3, and it is further ensured that the induced current generated by the induced electromotive force is not conducted to the head nail 3 so that the head nail 3 is prevented from heating up and the head of the patient is prevented from being burned. By using a separate insulating sleeve 4 to disable the conductive loop, not only the structure of the head nail 3 can be avoided from being changed and the durability of the head nail 3 can be ensured, but also the insulating sleeve 4 is not easily to be worn, compared with the insulating layer. Even if the insulating sleeve 4 is worn, because the insulating sleeve 4 is detachably connected with the vertical rod 2 and the head nail 3 and a new insulating sleeve 4 can be installed by a simple replacement, and the economic applicability of the positioning head frame is improved.

One skilled in the art will appreciate that the insulating sleeve 4 is also mounted on top of the vertical rod 2 in the radial direction of the base ring 1 and has a central through hole. The inner wall of the central through hole is provided with the inner thread and the outer wall of the head nail 3 is provided with an external thread section, and the inner and outer threads are engaged to achieve the threaded connection of the head nail 3 and the insulating sleeve 4. Also, the length of the external thread segment on the outer wall of the head nail 3 is longer than the internal thread length of the insulating sleeve 4 to ensure that the head nail 3 is able to abut against the target position of the patient's cranium by adjusting the threading depth of the head nail 3.

The insulating sleeve 4 is detachably connected with the top of the vertical rod 2, so that the insulating sleeve 4 can be disassembled and replaced in time after abrasion. It can be seen that under the premise that the connection and fastening of the insulating sleeve 4 and the vertical rod 2 are ensured, it is necessary to make the insulating sleeve 4 and the vertical rod 2 convenient to be detached. An example of the detachable connection mode of the insulating sleeve 4 and the vertical rod 2 is provided below:

(1) As a First Example:

According to the embodiment of the present disclosure, the insulating sleeve 4 is clamped to the top of the vertical rod 2, so that not only the connection and fastening can be ensured, but the disassembly is also convenient.

For the clamping manner, several illustrative examples are given below:

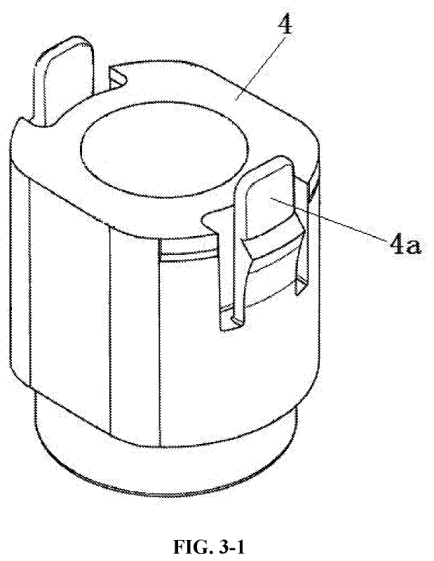

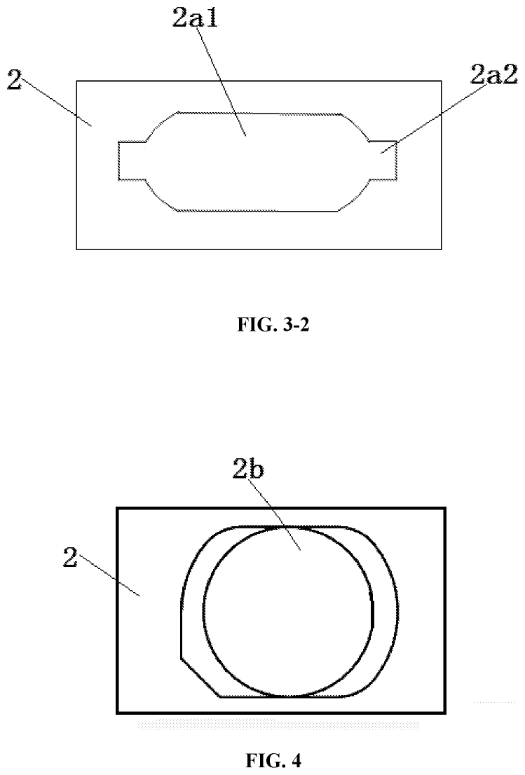

(1-1) As shown in FIG. 3-1, at least one elastic clip 4a is arranged on the side wall of the insulating sleeve 4;

As shown in FIGS. 3-2, the top of the vertical rod 2 is provided with a clamping hole 2a1, and a clamping groove 2a2 matching with the elastic clip 4a is arranged in the clamping hole 2a1;

The insulating sleeve 4 is detachably clamped in the clamping hole 2a1 through the elastic clip 4a.

In this embodiment, based on the elastic action of the elastic clip 4a, when sleeved into the clamping hole 2a1, the insulating sleeve 4 can be compressed by the hole wall of the clamping hole 2a1, and when moving to the clamping groove 2a2, the insulating sleeve 4 can be released, thereby clamping the insulating sleeve 4 into the clamping hole 2a1. When disassembled, the insulating sleeve 4 is pulled out of the clamping hole 2a1. In the pulling process, the elastic clip 4a is again compressed until it is pulled out from the clamping groove 2a2.

The elastic clip 4a may be provided in a variety of ways, for example, the elastic clip 4a may be provided in two opposite directions, and two opposed receiving grooves are formed in the side wall of the insulating sleeve 4 in an axial direction from one end, and one end of the elastic clip 4a is connected with the cut-off end of the accommodating groove. When the elastic clip 4a is compressed, the elastic clip 4a can be accommodated in the accommodating groove to achieve the effect of shorting the diameter of the insulating sleeve 4, so that the insulating sleeve 4 can enter or exit the clamping hole 2a1.

To ensure that the elastic clip 4a is clamped within the slot 2a2 and facilitates entry or exit of the card slot 2a2 under an external force. As shown in FIG. 3-1, the elastic clip 4a may include a resilient body disposed in the axial direction of the insulating sleeve 4, and a triangular protrusion disposed on the outer side of the elastic sheet in the circumferential direction of the insulating sleeve 4.

(1-2) A clamp matching with the insulating sleeve 4 is arranged on the top of the vertical rod 2, and the insulating sleeve 4 can be detachably connected with the vertical rod 2 by the clamp enclasping the insulating sleeve 4.

(2) As a Second Example:

As shown in FIG. 4, according to the embodiment of the present disclosure, a fixing hole 2b is provided at the top of the vertical rod 2 so that the insulating sleeve 4 in interference fit with the fixing hole 2b.

In this embodiment, the insulating sleeve 4 can be fixed in the fixing hole 2b or can be withdrawn from the fixing hole 2b by a thermal assembly process. Also, a tight connection may be ensured due to the interference fit of both.

(3) As a Third Example:

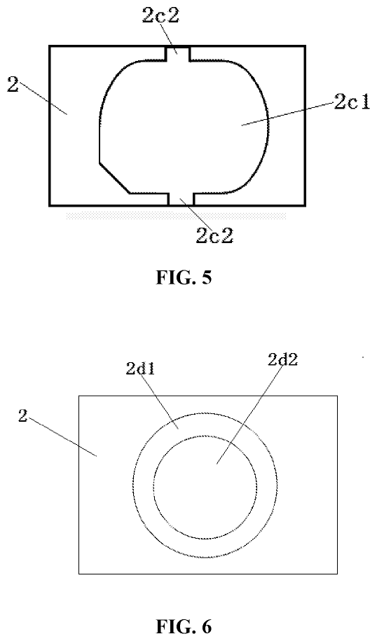

As shown in FIG. 5, the top of the vertical rod 2 is provided with a mounting hole 2c1 and a limiting hole 2c2 with the axes perpendicular to each other;

The insulating sleeve 4 is in clearance fit with the mounting hole 2c1;

The positioning head frame further includes a fastening screw which is in a threaded connection with the limiting hole 2c2 and is used for abutting against the insulating sleeve 4 after passing through the limiting hole 2c2.

When applied, the insulating sleeve 4 is placed in the mounting hole 2c1 and then passes through the limiting hole 2c2 by using the fastening screw in a manner of thread connection until the insulating sleeve 4 abuts against the outer wall of the insulating sleeve 4, and thus the insulating sleeve 4 is locked in the mounting hole 2c1. When it needs to be disassembled, what is required is only turning the fastening screw in the reverse direction to disengage it from the insulating sleeve 4.

In order to further ensure the tightness of the insulating sleeve 4, a positioning blind hole matching with the end of the fastening screw can also be provided on the outer wall of the insulating sleeve 4. When applied, the fastening screw passes through the limiting hole 2c2 in a threaded connection manner, and the end thereof is inserted into the positioning blind hole in the outer wall of the insulating sleeve 4, so that the stable installation of the insulating sleeve 4 in the mounting hole 2c1 can be ensured.

(4) As a Fourth Example:

As shown in FIG. 6, the top of the vertical rod 2 is provided with a large diameter hole 2d1 and a small diameter hole 2d2 which are coaxially connected.

The inner wall of the large diameter hole 2d1 is provided with an internal thread that rotates in the same direction as the internal thread of the insulating sleeve 4, and the insulating sleeve 4 is in threaded connection with the large diameter hole 2d1.

The diameter of the small diameter hole 2d2 is smaller than the outer diameter of the insulating sleeve 4, and is larger than the outer diameter of the head nail 3.

The insulating sleeve 4 is in threaded connection with the large diameter hole 2d1.

In application, the insulating sleeve 4 is first screwed into the large diameter hole 2d1. Since the diameter of the small diameter hole 2d2 is smaller than the diameter of the insulating sleeve 4, the insulating sleeve 4 is fixed in the large diameter hole 2d1 when the insulating sleeve 4 moves to abut against the outer wall of the small diameter hole 2d2. When disassembling, the insulating sleeve 4 can be screwed in a reverse direction.

Since the internal threads on the inner wall of the large diameter hole 2d1 rotate in the same direction as the internal threads of insulating sleeve 4, when the head nail 3 is screwed into the insulating sleeve 4, the position of the insulating sleeve 4 will remain unchanged. The head nail 3 can move axially along the insulating sleeve 4 until it is moved against the target position of the cranium of the patient.

As will be appreciated by those skilled in the art, in the various examples mentioned above, the outer profile of the insulating sleeve 4 matches with the structure of the corresponding mounting hole 2c1 to ensure that both are connected and fastened.

Further, to facilitate installation of the insulating sleeve 4, embodiments of the present disclosure may also improve the structure of the insulating sleeve 4, as shown in FIGS. 7-1 and 7-2, the insulating sleeve 4 includes a cylindrical section 401, and a force application section 402 connected to the cylindrical section 401.

The cylindrical section 401, which facilitates the molding process and has a high precision, is provided as a positioning reference when the insulating sleeve 4 is installed. It is understood that the top of the vertical rod 2 is provided with a mounting hole corresponding to the cylindrical section 401 and the force application section 402, respectively. By setting the force application section 402, the moment generated when the insulation sleeve 4 is screwed into or screwed out of the mounting hole on the top of the vertical rod 2 is born, and the component can ensure the connection precision of the head nail 3 and the vertical rod 2 and can ensure the reliability and the durability when the head nail 3 is stressed.

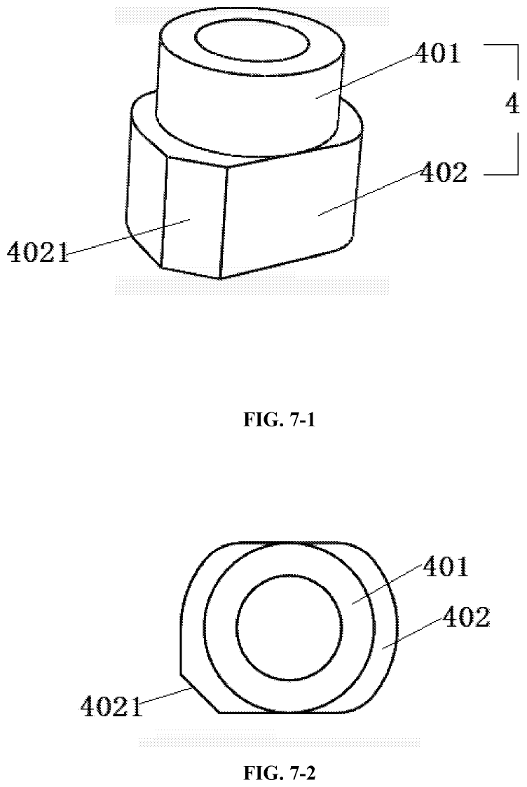

As an example, as shown in FIGS. 7-2, the force application section 402 is prepared by forming two opposing planes on a cylinder. Further, the diameter of the cylinder is larger than the diameter of the cylindrical section 401 such that the force application section 402 and the cylindrical section 401 form a step. When the insulation sleeve 4 works, the stress surface of the insulating sleeve 4 is the surface of the step, i.e., the step can bear the force from the axial direction of the head nail 3 and the stress performance of the insulation sleeve 4 is improved.

Typically, for a set of positioning head frame, it is often provided with a different length and configuration of vertical rods 2 to match different types of patients. At this time, the size, structure, and the like of the insulating sleeve 4 will vary with the vertical rod 2. In order to improve the fault tolerance rate of the insulation sleeve 4 during installation, as shown in FIGS. 7-1 and 7-2, embodiments of the present disclosure are provided with a fool-proof structure 4021 on a force application section 402.

The fool-proof structure 4021 is provided to ensure that the insulating sleeve 4 can only be mounted in the vertical rod 2 matching with the insulating sleeve 4. For example, the fool-proof structure 4021 may be obtained by providing a cross-section of a particular geometry on a sidewall of the force application section 402. Considering the purpose of excellent foolproof effect and simple preparation, the fool-proof structure 4021 may be in a rectangular cross-section arranged along the axial direction of the force application section 402.

As mentioned above, according to the embodiments of the present disclosure, the base ring 1 and/or the vertical rod 2 are insulated, i.e., the base ring 1 is insulated, or the vertical rod 2 is insulated, or the base ring 1 and vertical rod 2 are insulated at the same time. In these three embodiments, the conductive loop of the positioning head frame can be disabled.

On this basis, the base ring 1 can be wholly insulated or partially insulated in order to realize the insulation of the base ring 1 and the vertical rod 2; and/or the vertical rod 2 is wholly insulated or partially insulated.

Taking the vertical rod 2 as an example, when the vertical rod 2 is integrally insulated, the vertical rod 2 can be integrally formed by forming an insulating material. When the vertical rod 2 is partially insulated, it comprises at least an insulating segment formed of insulating material, i.e., the vertical rod 2 can be designed as a combination of metal segment and an insulating segment.

Likewise, the integral insulation or partial insulation formation of the base ring 1 is similar to the vertical rod 2 and will not be detailed herein.

Considering that the engineering plastic has the advantages of having high rigidity, small creep, high mechanical strength, good heat resistance, good electrical insulation property, capable of being used for a long time in harsh chemical and physical environment, and can replace metal as engineering structures and the like, engineering plastics can be used to impart insulating properties to the base ring 1 and the vertical rod 2, i.e., using engineering plastics to insulate the vertical rod 2 and/or the base ring 1 in whole or part.

When an engineering plastic is used for forming insulation, it may be connected with the metal section by an injection molding mode or by means of a bolt connection so as to ensure connection and fastening.

Further, the insulation performance and strength requirements of the positioning head frame are comprehensively considered, and the engineering plastic can be polyetheretherketone (PEEK) plastic, polyformaldehyde (POM) plastic and the like.

The foregoing descriptions are merely illustrative embodiments of the present disclosure, and are not intended to limit the present disclosure. Within the spirit and principles of the disclosure, any modifications, equivalent substitutions, improvements, etc., are within the protection scope of the present disclosure.

1. A gamma knife positioning head frame comprising:
a base ring;
a plurality of vertical rods circumferentially arranged on the base ring; and
a plurality of head nails in threaded connection with the plurality of vertical rods,
wherein at least one of the base ring or the vertical rod is insulated.

2. The positioning head frame according to claim 1, wherein the positioning head frame further comprises an insulating sleeve, the insulating sleeve is detachably connected with a top of the vertical rod, and an inner wall of the insulating sleeve is provided with internal threads and is in threaded connection with the head nail.

3. The positioning head frame according to claim 2, wherein the insulating sleeve is clamped to the top of the vertical rod.

4. The positioning head frame according to claim 3, wherein at least one elastic clip is arranged on a side wall of the insulating sleeve;
wherein a clamping hole is formed in the top of the vertical rod, and a clamping groove matching with the at least one elastic clip is arranged on an inner wall of the clamping hole; and
wherein the insulating sleeve is detachably clamped in the clamping hole by the at least one elastic clip.

5. The positioning head frame according to claim 2, wherein a fixing hole is arranged in the top of the vertical rod and the insulating sleeve is in interference fit with the fixing hole.

6. The positioning head frame according to claim 2, wherein the top of the vertical rod is provided with a mounting hole and a limiting hole, axes of the mounting hole and a limiting hole are perpendicular to each other and the insulating sleeve is in clearance fit with the mounting hole; and
wherein the positioning head frame further comprises a plurality of fastening screws in threaded connection with the limiting hole and configured for abutting against the insulating sleeve after passing through the limiting hole.

7. The positioning head frame according to claim 2, wherein the top of the vertical rod is provided with a large diameter hole and a small diameter hole which are in coaxial communication;
wherein an inner wall of the large diameter hole is provided with internal threads rotating in the same direction as internal threads of the insulating sleeve, and the insulating sleeve is in threaded connection with the large diameter hole, and
wherein a diameter of the small diameter hole is smaller than an outer diameter of the insulating sleeve and is larger than an outer diameter of the head nail.

8. The positioning head frame according to claim 2, wherein the insulating sleeve comprises a cylindrical section and a force application section connected to the cylindrical section.

9. The positioning head frame of claim 8, wherein the force application section has two opposed planes and a diameter of the cylinder is larger than a diameter of the cylindrical section.

10. The positioning head frame according to claim 8, wherein the force application section is provided with a fool-proof structure.

11. The positioning head frame according to claim 1, wherein at least one of the base ring and the vertical rod is wholly or partially insulated.

12. The positioning head frame according to claim 11, wherein at least one of the vertical rod or the base ring is wholly or partially insulated, and material of an insulated portion of the vertical rod and the base ring is engineering plastic.
